(19) United States
(12) Patent Application Publication
(10) Pub. No.: US 2010/0069856 A1
(43) Pub. Date: Mar. 18, 2010

(54) Structure for Connecting between a Medical Needle and Medical Tubing

(75) Inventors: Yosuke SAKAI, Shizuoka (JP); Aya TSUKANAKA, Shizuoka (JP); Takashi SATOH, Shizuoka (JP); Taiki ATSUMI, Shizuoka (JP)

Correspondence Address: TYCO HEALTHCARE GROUP LP, 15 HAMPSHIRE STREET, MANSFIELD, MA, 02048, US

(73) Assignee: TYCO HEALTHCARE GROUP LP, Mansfield, MA (US)

(21) Appl. No.: 12/557,793

(22) Filed: Sep. 11, 2009

(30) Foreign Application Priority Data
Sep. 12, 2008 (JP) 2008-234272

(51) Int. Cl. A61M 39/10; A61M 5/00

(52) U.S. Cl. 604/272; 604/533

(57) ABSTRACT

A connecting structure is provided which includes a medical needle with a connector provided with a joining groove formed on its rear-end portion, an inner tube that joins to the joining groove, and medical tubing that has one end secured to the outer peripheral surface of the inner tube by welding or adhesive. Moreover, the connector and the inner tube are shaped so as to form a cylinder when the inner tube is fit to the joining groove of the connector. In addition, a portion that is not covered by the inner tube is provided at the base-end portion of the connector, thus providing a gap between the base-end portion of the connector and the medical tubing.

### TECHNICAL FIELD

The present disclosure relates to the structure of a connection between a medical needle and medical tubing that is left in a wound after surgery.

### BACKGROUND

Conventionally, after surgery, medical tubing is inserted into a wound to perform the drainage of blood or other exudates that may arise in the wound. In such a case, a medical needle is attached to the tip of the medical tubing, and the medical needle is passed from the wound toward the outside of the body of the patient, thus leaving the rear-end portion of the medical tubing in the wound. Moreover, after removing the medical needle from the tip of the medical tubing, the tip of a tube extending from a storage container via a pump is connected to this tip and the pump is operated to suck the blood or other exudates from the wound into the interior of the storage container, as illustrated for example, in Japanese Patent Publication No. JP H2-17185, the entire contents of which is hereby incorporated by reference.

This wound drain catheter, or medical tubing is connected to a trocar, or medical needle via a drain tube constituting the tip portion, and a connector portion is formed at the rear end of the trocar where the drain tube is connected. This connector portion is provided with a plurality of annular protrusions, and these protrusions are joined in a grasping manner to the inside wall of the drain tube, and thus the wound drain catheter is connected to the trocar.

However, the conventional wound drain catheter is connected in a connection portion where a plurality of annular protrusions is formed, so the shape of the steps in the protrusions is exposed on the outside surface of the wound drain catheter, and so the surface of the wound drain catheter is uneven. For this reason, greater resistance arises when piercing the wound drain catheter into the body of the patient, so smooth handling becomes more difficult. In addition, adhesive or the like is used for connecting to the medical tubing at a cylindrical connecting portion, thus it is difficult to maintain adequate adhesion. Accordingly, additional costs would be required to achieve adequate adhesion.

### SUMMARY

A structure for connecting between a medical needle and medical tubing is provided which includes a medical needle wherein a connector provided with a joint is formed upon its rear end; a conjugating member that is joined to said joint in a state such that it covers at least part of said connector, and is formed in a cylindrical shape at said connector when it covers at least part of said connector; and medical tubing that is connected to said medical needle by covering a cylindrical body made up of said connector and said conjugating member in a state in which the inside surface at one end is secured to the outside surface of said conjugating member.

### BRIEF DESCRIPTION OF THE DRAWINGS

Various embodiments of the present disclosure will be described herein below with reference to the figures wherein:

FIG. 1 is a partial-cutaway side view showing the connecting structure according to an embodiment of the present disclosure;

FIG. 2 is a cross section of the main portion of the connecting structure of the present disclosure;

FIG. 3 is a cross section of the main portion of the connecting structure according to an embodiment of the present disclosure; and

FIG. 4 is a perspective view showing the split member of the connecting structure of the present disclosure.

### DETAILED DESCRIPTION

The present disclosure provides a structure for connecting between a medical needle and medical tubing wherein no unevenness occurs in the connecting portion between the medical needle and medical tubing, and moreover its cost is low.

In embodiments, the structure for connecting between a medical needle and medical tubing according to the present disclosure may include a medical needle wherein a connector provided with a joint may be formed upon its rear end; a conjugating member that may be joined to the joint in a state such that it may cover at least part of the connector, and may be formed in a cylindrical shape at the connector when it covers at least part of the connector; and medical tubing that may be connected to the medical needle by covering a cylindrical body made up of the connector and the conjugating member in a state in which the inside surface at one end may be secured by deposition or adhesion to the outside surface of the conjugating member.

In embodiments, the structure for connecting between a medical needle and medical tubing according to the present disclosure does not provide unevenness at the connector to the medical needle, thus joining the medical tubing at the uneven connector, but rather at least part of the connector may be covered with a conjugating member that is able to join to the connector, thus causing the union between the connector and the conjugating member to be formed in a cylindrical shape. Moreover, one end of the medical tubing may be secured to the outside surface of the cylindrically formed connector and conjugating member. In embodiments, the conjugating member and medical tubing may be secured by welding or adhesion. To wit, utilized to form the conjugating member may be selected such that it can be secured to the medical tubing by welding or adhesion. Thereby, there is no unevenness on the outside surface of the union between the connector and conjugating member located in the interior of the medical tubing, such that no unevenness occurs on the surface of the medical tubing either. As a result, the medical tubing may exhibit little resistance when piercing the body of the patient, so smooth handling may become possible.

In embodiments, the structure for connecting between a medical needle and medical tubing according to the present disclosure may include a connector formed in a cylindrical shape, wherein the joint may be constituted with a groove formed on the outside surface of the connector along the circumference thereof, the conjugating member may be constituted with an elastic cylindrical body that joins to the groove, and the thickness of the conjugating member may be set to be approximately the same as the depth of the groove.

In embodiments, the conjugating member may be made of a material that is elastic, such that even if the conjugating member is made from a single cylindrical body, the conjugating member may be joined to the groove by flexing or stretching. Moreover, the conjugating member may be restored to its original shape after joining to the groove. At this time, the thickness of the conjugating member may be set to be roughly the same as the depth of the groove, such that the outside surface of the connection and the outside surface of the conjugating member may be connected so as to be positioned upon the same surface, and thus the medical tube may be easily fitted to the connector and conjugating member.

In embodiments, it may be possible to provide a slit in the connecting material that extends in the axial direction, so that this slit may be opened and closed. Thereby, the conjugating member may be fitted to the connector in the state in which the slit is open, and then, after fitting to the connector, the conjugating member may be returned to its original cylindrical shape.

In embodiments, the structure for connecting between a medical needle and medical tubing according to the present disclosure may include a connector formed in a columnar shape, wherein the joint may be constituted with a flange formed on the outside surface of the connector so as to protrude outward thereupon, and the conjugating member may be formed with a cylindrical body provided with a groove that is able to join to the joint. Thereby, movement of the conjugating member with respect to the medical needle in the direction of the axis of the medical tube may be effectively prevented, so it may be possible to prevent the conjugating member and medical tubing from coming off of the medical needle. In embodiments, the connector of the present disclosure may be formed in the shape of a circular column, triangular column, square column, polygonal column, and other columnar shapes.

In embodiments, the structure for connecting between a medical needle and medical tubing according to the present disclosure may include a conjugating member formed with a plurality of members that are divided in the direction around the axis of the cylindrical body. Thereby, the individual members constituting the conjugating member may be sequentially disposed around the connector, thus simplifying the work of fitting the conjugating member to the connector.

In embodiments, the structure for connecting between a medical needle and medical tubing according to the present disclosure may include a portion not covered with the conjugating member provided at the base end of the connector, and a gap may be provided between the base end of the connector and the medical tubing. Thereby, it may be possible to prevent the outer peripheral edge of the opening of the medical tubing from floating up on the outer periphery side. The gap should be of a depth that can prevent the end of the medical tubing from being pushed open by the connector toward the outer peripheral side.

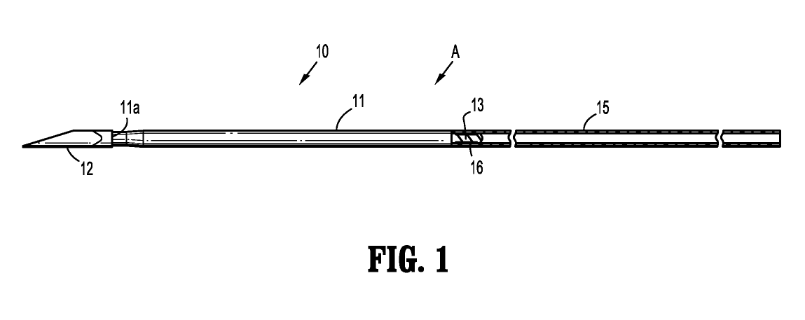

FIG. 1 shows the connecting structure A according to an embodiment of the present disclosure, having a medical needle 10, medical tubing 15 and an inner tube 16 serving as the conjugating member according to the present disclosure. Medical needle 10 includes a cylindrical needle body 11 with a small diameter, a puncturing portion 12 formed at the tip of the needle body 11 and a connector 13 formed at the rear end of the needle body 11. In addition, a narrow portion is formed at the boundary between the needle body 11 and the puncturing portion 12, where the diameter is gradually made smaller when heading from the needle body 11 toward the puncturing portion 12 and thereafter kept at the same diameter, and a joining step 11a is formed at the front end thereof.

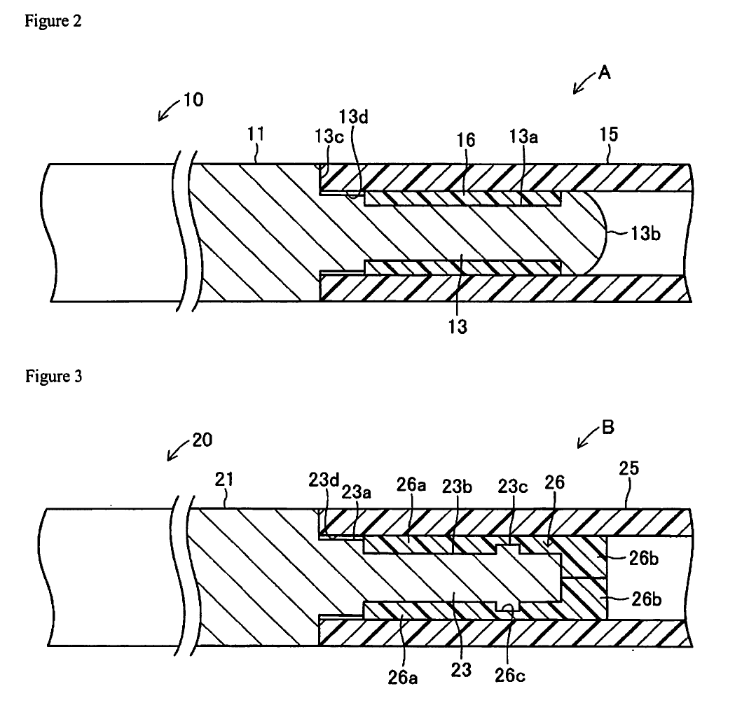

As shown in FIG. 2, the connector 13 is provided in order to connect the medical tubing 15 to the medical needle 10, being formed roughly in the shape of a cylinder with a diameter smaller than that of the needle body 11. A wide joining groove 13a constituting the joint according to the present disclosure is formed in the peripheral surface of this connector 13, in portions excluding the portions at either end in the axial direction, and the rear-end surface 13b of the connector 13 is formed in the shape of a smooth convex curved surface. In addition, a step 13c which includes a surface that is perpendicular to the axial direction of the medical needle 10 is formed between the needle body 11 and the connector 13, and the diameter of the portion between the joining groove 13a and the step 13c on the outer peripheral surface of the connector 13 is set to be somewhat smaller than the diameter of the rear-end portion of the connector 13 (the portion towards the right in FIG. 2).

The inner tube 16 is formed of a cylinder that is provided with flexibility and elasticity, and can be fit within the joining groove 13a of the connector 13 by pushing forward with its opening positioned at the rear-end surface 13b of the connector 13. In this case, the rear-end surface 13b is formed in the shape of a smooth convex curved surface, so the work of fitting the inner tube 16 within the joining groove 13a can be performed smoothly. Moreover, when the inner tube 16 is fitted within the joining groove 13a, the outer peripheral surface of the inner tube 16 and the outer peripheral surface of the rear-end portion of the connector 13 form a continuous curved surface with no difference in height. In this case, a small amount of difference in height may occur. In addition, a slight difference in height is formed between the outer peripheral surface of the inner tube 16 and the outer peripheral surface of the base-end portion of the connector 13. This inner tube 16 may be made of polyvinyl chloride or polyurethane.

The medical tubing 15 may be made of polyvinyl chloride or polyurethane, having an inside diameter such that it can cover the outside of the inner tube 16 and connector 13 which forms a cylinder with the inner tube 16 inserted within the joining groove 13a. Medical tubing 15 may be fit to the outside of the connector 13 with the inner tube 16 fit on, by pushing forward with its opening positioned at the rear-end surface 13b of the connector 13, and may be secured to the inner tube 16 by adhesive or welding.

The rear-end surface 13b is formed in the shape of a smooth convex curved surface, so the work of fitting the medical tubing 15 to the connector 13 can be performed smoothly. When the medical tubing 15 is fitted to the connector 13, a gap 13d is formed between the medical tubing 15 and the outside peripheral surface of the base-end portion of the connector 13, while the medical tubing 15 is in close contact with the outside peripheral surface of the rear-end portion of the connector 13. In addition, the end surface of the medical tubing 15 is in contact with the step 13c of the medical needle 10 in the state such that the outside peripheral edge is prevented from protruding from the outside peripheral edge toward the outside.

Suitable adhesives for securing the medical tubing 15 to the inner tube 16 may include ultraviolet-curing adhesives, visible light-curing adhesives, instant adhesives, or the like. When the medical tubing 15 is to be secured to the inner tube 16 by welding, methods such as, for example, the solvent welding method and the heat welding method may be utilized. Suitable solvents utilized in the solvent welding method may include cyclohexane, tetrahydrofuran, and the like. When an adhesive or solvent is to be used, the inner tube 16 and the medical tubing 15 are assembled in a state such that the outside peripheral surface of the inner tube 16 or the inside peripheral surface of the medical tubing 15 is coated with an adhesive or a solvent.

For example, when utilizing the heat welding method, the medical needle 10 is heated to a temperature of 200° C., for example, using a stipulated heating apparatus and heat-shrinking tube. In this case, first the connecting portion of the medical tubing 15 and inner tube 16 is covered with a heat-shrinking tube. Then, the portion of the medical tubing 15 and inner tube 16 covered with the heat-shrinking tube is heated. Next, the connecting portion of the medical tubing 15 and inner tube 16 is melted by heating and unitized and then that portion is cooled. Thereby, the welding of the medical tubing 15 to the inner tube 16 is complete. Then, the heat-shrinking tube is removed from the welded medical tubing 15 and inner tube 16. The connecting structure A shown in FIG. 1 can thus be obtained by these methods.

With this structure, when the medical tubing 15 is to be inserted into a wound (not shown) of a patient, first the medical needle 10 with the connecting structure A is inserted from an incision in the body of the patient toward the wound, and then the puncturing portion 12 at the tip is brought outside the body from the stipulated portion. At the time of this puncture, if necessary, it is possible to bend appropriate portions of the needle body 11 so that the medical needle 10 can puncture the appropriate position of the wound. In this case, the surface of the connector between the medical needle 10 and the medical tubing 15 includes a smooth curved surface with no unevenness, so the resistance at the time of piercing is reduced so smooth operation becomes possible. In addition, a gap 13d is formed between the medical tubing 15 and the outer peripheral surface of the base-end portion of the connector 13, so the tip of the medical tubing 15 does not protrude toward the outside.

This prevents the tip of the medical tubing 15 from protruding toward the outside and interfering with the piercing. Moreover, once the puncturing portion 12 of the medical needle 10 has left the body, it is possible to attach a stipulated withdrawal tool (not shown) to the joining step 11a positioned at the rear of the puncturing portion 12, and thus manually hold the withdrawal tool and pull to draw the medical needle 10 outside of the body. Once the tip end of the medical tubing 15 comes out of the body, and the rear end of the medical tubing 15 is positioned at the wound, the pulling operation is halted and scissors are used to cut off the medical needle 10 and the connector-side portion of the medical tubing 15. Next, the cut portion of the medical tubing 15 is connected to a stipulated suction pump (not shown). The operation of the suction pump sucks out the blood and other exudates that collect in the wound via the medical tubing 15.

In this manner, a cylindrical inner tube 16 is fitted to the joining groove 13a of the connector 13, and thus the connector 13 and inner tube 16 form a cylindrical shape. Then, the cylindrically formed outer peripheral surface of the connector 13 and inner tube 16 are covered with one end of the medical tubing 15, and the inner tube 16 is secured to the medical tubing 15 by welding or adhesive. Thereby, there is no unevenness in the outer peripheral surface of the connector 13 and inner tube 16 at their position in the interior of the medical tubing 15, so no unevenness occurs in the surface of the medical tubing 15 either. As a result, the resistance is reduced at the time that the medical needle 10 is pierced through the body of the patient, and smooth handling is possible.

In addition, a gap 13d is formed between the medical tubing 15 and the outside peripheral surface of the base-end portion of the connector 13, so the tip of the medical tubing 15 does not protrude from the connector 13 toward the outside. Thereby, the piercing by the medical needle 10 can be performed even more smoothly. The joint of the connector 13 forms a joining groove 13a and the joining member forms a cylindrical inner tube 16 which is joined to the joining groove 13a, and thus it is possible to obtain a connecting structure A with a simple constitution where no unevenness occurs in the connecting portion.

FIG. 2 shows a connecting structure B according to another embodiment of the present disclosure. With connecting structure B, connector 23 of medical needle 20 is formed in the shape of a stepped rod with a circular cross section of a diameter smaller than that of the needle body 21. To wit, connector 23 includes a cylindrical large-diameter portion 23a positioned at the base-end side with a short length in the axial direction, and a cylindrical small-diameter portion 23b that is concentric to the large-diameter portion 23a, is positioned at the rear-end side of the large-diameter portion 23a, which has a longer length in the axial direction and has a diameter smaller than that of the large-diameter portion 23a. Moreover, a flange-shaped joint 23c that protrudes toward the outside along the circumference of the rear-end side portion of the small-diameter portion 23b (the portion toward the right side of FIG. 2). This joint 23c protrudes from the outer peripheral surface of the small-diameter portion 23b by a distance that somewhat greater than half of the distance between the rear edge of the outer peripheral surface of the large-diameter portion 23a and the front edge of the outer peripheral surface of the small-diameter portion 23b. In addition, the rear-end surface of the small-diameter portion 23b is formed as a plane.

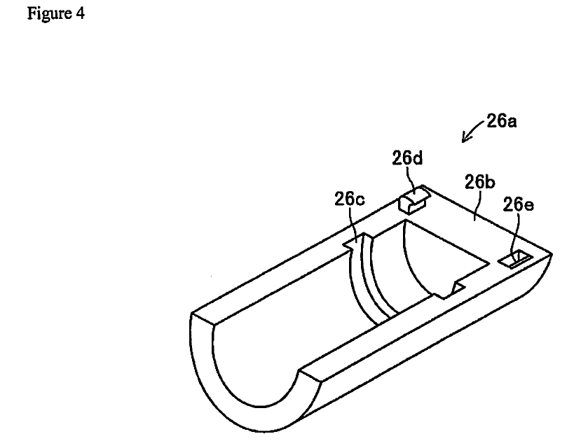

A conjugating molded form 26 is attached to the small-diameter portion 23b over its outer peripheral surface and rear-end surface. The conjugating molded form 26 includes two split members 26a of the same shape that are made of hard materials, so by assembling the two split members 26a, a cylindrical form with its rear end portion closed is formed. As shown in FIG. 4, each of the split members 26a is formed in the shape of a semicylindrical gutter with one end closed by a semicircular plate-shaped wall 26b, and a joint groove 26c is formed, on the inner peripheral surface toward the side of the wall 26b from the center in the axial direction, in the peripheral direction so as to be able to join to one half of the joint 23c along its periphery. Moreover, a joining prong 26d is formed on one side of the meeting surface (the upper surface in FIG. 4) of the wall 26b, and a joining hole 26e is formed on the other side of the meeting surface of the wall 26b so that it is able to join to the joining prong 26d of the other of the split members 26a.

Thus, when the two split members 26a are fitted to the outer periphery of the small-diameter portion 23b of the connector 23 with the two joint grooves 26c each joined to the joint 23c and also with the joining prongs 26d and joining holes 26e joined to each other, the small-diameter portion 23b and joint 23c are covered by the two split members 26a. As a result, only the outer peripheral surface and end surface of the conjugating molded form 26 made up of two split members 26a appears at the rear-end portion of the medical needle 20. The thickness of the cylindrical portion of the conjugating molded form 26 is set to be roughly twice the length of protrusion of the joint 23c from the outer peripheral surface of the small-diameter portion 23b, and for this reason, a slight step is formed between the outer peripheral surface of the conjugating molded form 26 and the outer peripheral surface of the large-diameter portion 23a of the connector 23. Suitable materials for forming the conjugating molded form 26 may include polyvinyl chloride, polyurethane, polycarbonate, acrylonitrile-butadiene-styrene copolymer resin, combinations thereof, and the like.

The medical tubing 25 is secured to the conjugating molded form 26 by adhesive or welding, and a gap 23d is formed between the medical tubing 25 and the outside peripheral surface of the large-diameter portion 23a of the connector 13. In addition, the end surface of the medical tubing 25 is in contact with the step between the needle body 21 and the large-diameter portion 23a in the state such that the outside peripheral edge is prevented from protruding from the outside peripheral edge of the needle body 21 toward the outside. The other portions of connecting structure B are formed in the same manner as those of the aforementioned connecting structure A. In addition, the method of using the medical needle 20 and medical tubing 25 provided with connecting structure B is identical to the method of using the aforementioned connecting structure A.

Therefore, movement of the conjugating molded form 26 and medical tubing 25 with respect to the medical needle 20 in the direction of the axis of the medical tube can be effectively prevented, so it is possible to prevent the conjugating molded form 26 and medical tubing 25 from coming off of the medical needle 20. In addition, the conjugating molded form 26 includes two split members 26a, so the conjugating molded form 26 can be easily fit to the small-diameter portion 23b. Moreover, with connecting structure B, the joint 23c of the connector 23 is embedded within the joint groove 26c of the conjugating molded form 26 to a depth of roughly ½ of the wall thickness of the conjugating molded form 26, so the conjugating molded form 26 can be fit to the connector 23 while maintaining the strength of the conjugating molded form 26.

It will be appreciated that variations of the above-disclosed and other features and functions, or alternatives thereof, may be desirably combined into many other different systems or applications. Also that various presently unforeseen or unanticipated alternatives, modifications, variations or improvements therein may be subsequently made by those skilled in the art which are also intended to be encompassed by the following claims. Unless specifically recited in a claim, steps or components of claims should not be implied or imported from the specification or any other claims as to any particular order, number, position, size, shape, angle, color, or material.

For example, in an embodiment, the cross-sectional shape of the connector 23 is set to be round, but the cross-sectional shape of the connector 23 need not be round, but rather it may also be triangular, square or another polygonal shape. In addition, the conjugating molded form 26 takes the form of a cylinder with the end closed, but this conjugating molded form 26 may also constitute two or more split members.

In alternative embodiments, while the inner tube 16 includes a heat-shrinking tube that is shrunk by heating and then cooling, it may also be secured to the connector 13 or 23 by being fitted to the connector 13 or 23 and then heated and cooled. In this case, the inner tube 16 and medical tubing 15 may be made of polyvinyl chloride. Moreover, it is also possible to provide a slit extending in the axial direction of the inner tube 16, and permit this slit to open and close. In this case, the conjugating member may be fit to the connector in the state with the slit open and after fitting to the connector, the conjugating member is restored to its original cylindrical shape.

What is claimed is:

1. A connecting structure comprising:
a medical needle having a connector with a joint formed upon its rear end;
a conjugating member joined to said joint in a state such that it covers at least part of said connector, and having a cylindrical shape at said connector when it covers at least part of said connector; and
medical tubing connected to said medical needle by covering a cylindrical body made up of said connector and said conjugating member in a state in which an inside surface at one end is secured to an outside surface of said conjugating member.

2. The connecting structure according to claim 1, wherein said connector has a cylindrical shape.

3. The connecting structure according to claim 1, wherein said joint includes a groove formed on an outside surface of said connector along a circumference thereof.

4. The connecting structure according to claim 3, wherein said conjugating member is formed with an elastic cylindrical body that joins to said groove.

5. The connecting structure according to claim 1, wherein a thickness of said conjugating member configured to be approximately the same as a depth of said groove.

6. The connecting structure according to claim 1, wherein said connector is formed in a columnar shape.

7. The connecting structure according to claim 1, wherein said joint includes a flange formed on the outside surface of said connector so as to protrude outward thereupon.

8. The connecting structure according to claim 1, wherein said conjugating member includes a cylindrical body provided with a groove configured to join to said joint.

9. The connecting structure according to claim 4, wherein said conjugating member includes a plurality of members that are divided in a direction around an axis of the cylindrical body.

10. The connecting structure according to claim 1, wherein a portion not covered with said conjugating member is provided at a base end of said connector, and a gap is provided between the base end of said connector and said medical tubing.

11. The connecting structure according to claim 1, wherein the medical tubing is secured to an outer peripheral surface of an inner tube by an adhesive.

12. The connecting structure according to claim 1, wherein the medical tubing is secured to an outer peripheral surface of an inner tube by a weld.
